(19) United States
(12) Patent Application Publication
(10) Pub. No.: US 2004/0167480 A1
(43) Pub. Date: Aug. 26, 2004

(54) Administration of multiple viscoelastic solutions with a multi-compartment syringe

(75) Inventor: Gilles Bos, Ettlingen (DE)

Correspondence Address: Nicole S. Bradley, PILLSBURY WINTHROP LLP, Suite 2800, 725 South Figueroa Street, Los Angeles, CA, 90017, US

(73) Assignee: Advanced Medical Optics, Inc., Santa Ana, CA

(21) Appl. No.: 10/372,055

(22) Filed: Feb. 21, 2003

(51) Int. Cl. A61M 35/00

(52) U.S. Cl. 604/289

(57) ABSTRACT

A novel ophthalmic viscosurgical system and method is disclosed. The system and method comprise multiple viscoelastic solutions that are pre-selected based on desired characteristics. Each solution is placed in distinct locations within the eye during surgery to maintain the natural shape of the eye and facilitate the surgical procedure. Novel viscoelastic solutions for use in association with the present invention are also disclosed.

### FIELD OF THE INVENTION

The present invention relates to a method for conducting ophthalmic surgery. In particular, the invention relates to the concurrent use of multiple viscoelastic solutions during intraocular surgery, said solutions being optimally administered with a multi-compartment syringe.

### BACKGROUND OF THE INVENTION

The term viscosurgery refers to the use of viscoelastic substances in surgery. Ophthalmic viscosurgery encompasses a broad range of intraocular surgical procedures including, for example, cataract surgery, cornea surgery, glaucoma surgery, vitreous surgery, trauma surgery, intraocular lens implantation and retinal detachment surgery. Ophthalmic viscosurgical procedures frequently involve forming an incision in the cornea and subsequently gaining instrument access to one or all of the anterior chamber, lens capsular bag and posterior chamber for the purpose of manipulating target eye structures.

During ophthalmic viscosurgery, viscoelastic solutions are often introduced into the eye to protect tissues and to ease both the introduction of instruments into the surgical field and the concomitant manipulation of eye tissues. Some of the tasks performed by viscoelastics during ophthalmic surgery include lubrication, the coating of surfaces, the creation and maintenance of eye chamber spaces, the separation of tissues, the displacement of mobile tissue, the blocking of outflow of ocular fluids, the confinement of bleeding, the exclusion of inflammatory materials and the dampening of instrument movement in the eye. Importantly for present purposes, viscoelastics coat the chambers of the eye and, thus, accomplish the protection of sensitive tissues from trauma, particularly the corneal endothelium. In addition, viscoelastics restore the shape and depth of eye structures such as the anterior chamber and the lens capsular bag after the structures have been deflated by the loss of natural fluid volume following the formation of surgical incisions.

Cataract surgery is a routine ophthalmic viscosurgical procedure. The procedure can be divided into several steps. Following pupil dilation and local anesthesia, an incision is made through the cornea into the anterior chamber of the eye. Once the anterior chamber is punctured, the natural aqueous humor leaks out and the chamber loses its characteristic shape and depth. A viscoelastic product is then introduced into the anterior chamber so that the chamber regains its former volume and so that vulnerable tissues such as the corneal endothelium are protected. Next, a capsulotomy is performed, which involves making a hole in the anterior portion of the lens capsular bag. Extraction of the lens itself is then most often accomplished by phacoemulsification (disintegration of the lens by ultrasound, followed by aspiration of the resultant lens particles) or by extracapsular cataract extraction (removal of the lens in one piece). At this stage, a viscoelastic product is again needed to maintain the natural volume of the capsular bag and to facilitate the introduction of a synthetic lens. Upon completion of the surgery, the viscoelastic compound is removed from the eye and replaced with a physiologically compatible salt solution. Generally, viscoelastics cannot be left in the eye due to the compounds' viscosity and the eye's resultant inability to process the compounds through its filtering system. Leftover viscoelastics that cannot be adequately processed by the eye cause a detrimental increase in intraocular pressure.

Viscoelastics are solutions that have viscous, elastic and pseudoplastic properties. Viscoelastics, by definition, have dual properties. They act as both viscous liquids and as elastic solids or gels. The typical viscoelastic is an aqueous solution containing a polysaccharide such as sodium hyaluronate, chondroitin sulfate or hydroxypropylmethylcellulose. Concentrations of viscoelastics vary from 10-70 mg/ml and molecular masses (expressed as mass average relative molecular masses) vary from 20,000 daltons (chondroitin sulfate) to 5,000,000 (sodium hyaluronate). The most common viscoelastic used in ophthalmic surgery is Healon™ (Pharmacia, Inc., Piscataway, N.J.), a hyaluronic acid composition. The source of most commercially available hyaluronate derivative viscoelastics, including Healon™ (Pharmacia, Inc., Piscataway, N.J.), is the dermis of rooster combs. However, others manufacture products from sodium hyaluronate that are derived from a bacterial fermentation process.

There are two main classes of viscoelastic compounds: dispersive and cohesive. Viscoat™ (Alcon Laboratories, Inc., Forth Worth, Tex.) is an example of an dispersive compound while Healon™ (Pharmacia, Inc., Piscataway, N.J.) is an example of a cohesive compound. Dispersive products are typically high concentration (20 mg/ml to 70 mg/mL) solutions of low molecular weight (less than 500,000 daltons) polysaccharides. Cohesive products are typically low concentration (10-20 mg/mL) solutions of high molecular weight (1 to 5 million daltons) polysaccharides.

Dispersive viscoelastics usually show more adhesive properties and are effective agents for coating the corneal endothelium. Dispersive solutions resist washout during surgical procedures such as phacoemulsification, thus conferring lasting protection of endothelial tissues. However, dispersive viscoelastics are difficult to remove from eye at the conclusion of surgery due to their adhesive and viscous characteristics.

In contrast, cohesive viscoelastics have good space-maintaining properties. Cohesive products are good candidates for the displacement and stabilization of tissues. However, the cohesiveness of these products also produces negative characteristics. By definition, cohesive viscoelastics tend to stick to themselves rather than to adhere to tissue surfaces. Thus, they may leave the eye too quickly during surgery (for example, they may be displaced by phacoemulsification). Furthermore, they may leave the eye as a singular unit, a result that necessitates the reintroduction of a fresh volume of solution so as to maintain the eye chamber spaces.

It has been suggested to use more than one type of viscoelastic solution during ophthalmic surgery. For example, U.S. Pat. No. 5,273,056 (“the '056 patent”) suggests the use of different viscoelastic products in the various steps of cataract surgery.

Specifically, the '056 patent suggests the use of Viscoat™ (Alcon Laboratories, Inc., Forth Worth, Tex.) during the early stages of cataract surgery (before the capsulotomy) and the subsequent use of a high molecular weight hyaluronate composition, such as Healon™ (Pharmacia, Inc., Piscataway, N.J.), to maintain the shape and depth of the capsular bag during implantation of the synthetic lens. However, the '056 patent does not propose a sufficient strategy for addressing the disparate viscoelastic needs of the corneal endothelium and facilitating the movements of instruments in the anterior chamber during cataract surgery. As indicated above, dispersive viscoelastics are better suited for the task of coating the corneal endothelium, while cohesive viscoelastics are better suited for space maintenance in the remainder of the anterior chamber. The '056 patent suggests the use of Viscoat™ (Alcon Laboratories, Inc., Forth Worth, Tex.), a solution that possesses both adhesive and dispersive attributes, to fulfill both tasks: protecting the corneal endothelium and filling the anterior chamber.

Somewhat similarly, U.S. Pat. No. 6,368,585 teaches the use of a single viscoelastic solution in cataract surgery that seeks to achieve a balance between adhesive and cohesive characteristics. The system of the '585 patent has the distinct disadvantage of failing to fully satisfy any of the demands that are placed on viscoelastic solutions during the various stages of cataract surgery.

Several systems have been proposed for the delivery of viscoelastic solutions to the eye during ophthalmic surgery. For example, U.S. Pat. 6,254,587 B1 discloses a phacoemulsification handpiece that can perform many of the steps involved in cataract surgery. The device has a needle tip that can puncture the cornea as well as deliver both an irrigation fluid and a viscoelastic solution to the eye during surgery, either separately or as a mixed compound. However, the phacoemulsification device of the '587 patent and other similar devices do not have the capability to deliver multiple viscoelastic agents to the eye or to preferentially administer a viscoelastic to the inner surface of the cornea in an effective manner. Alternatively, U.S. Pat. No. 5,372,586 discloses a bi-compartment syringe that could be used to deliver two different viscoelastic agents. However, the device of the '586 patent requires the two compounds to be mixed in one part of the syringe before administration, thus making it impossible to preferentially administer different viscoelastic solutions to distinct regions of the eye during surgery, so as to properly confer the beneficial characteristics of each of the solutions.

Thus, the prior art does not achieve the most desirable outcome of providing a set of viscoelastic solutions that can be used concurrently to fully protect the corneal endothelium, to maintain the tissues and natural volume of the anterior chamber and lens capsular bag and to facilitate instrument movement during the execution of the capsular rhexis and phacoemulsification. Likewise, the prior art does not suggest the use, in ophthalmic surgery, of a multi-compartment syringe assembly that is capable of maintaining full separation between multiple viscoelastic solutions before preferentially administering the solutions to various areas of the eye and that does not require any significant mixing of the solutions prior to administration. In light of the disparate viscoelastic needs of the various regions of the eye during ophthalmic surgery, it is evident that such a syringe assembly would be desirable.

### BRIEF DESCRIPTION OF THE DRAWINGS

FIG. 1 shows a cross-sectional view of an eye; and

FIG. 2 shows a perspective view of a multi-compartment syringe according to an embodiment of the present invention.

### DETAILED DESCRIPTION OF THE INVENTION

It has been discovered that ophthalmic viscosurgical procedures can best be accomplished through the use of multiple viscoelastic solutions wherein one solution is applied in one area, and a second solution is applied to a different area. For example, one solution may be used to coat and protect the corneal endothelium while another solution may be used for the maintenance of the shape of the anterior chamber. Alternatively, or additionally, the second or even a third solution may be used for filling the lens capsular bag.

As discussed in more detail below, various different viscoelastic solutions are suitable for use in association with the present invention. Such solutions are chosen at least based in part on their viscosity and tendency to adhere within the eye. In one embodiment of the invention, viscoelastic solutions that can be completely removed from the eye following surgery are used. Viscoelastic solutions that cannot be completely removed from the eye may be used in association with the present invention, but are typically not desirable if they cannot be adequately processed by the eye and, thus, cause a detrimental increase in intraocular pressure. Of course, solutions having the desired physical properties which also may be adequately processed by the eye may be used in association with the present invention, and left in the eye.

Accordingly, a set of viscoelastic solutions is provided, wherein each solution is particularly suited for a particular use to in an ophthalmic viscosurgical procedure. For example, in one embodiment of the present invention, the first of the set of viscoelastic solutions is designed to coat and protect the corneal endothelium. The first of the set of viscoelastic solutions adheres to the inner surface of the cornea and is sufficiently tacky and robust so as to make it suited to the task of protecting the cornea. The second of the set of viscoelastics is designed to maintain the shape and depth of the anterior chamber, to facilitate instrument movement and the execution of the capsular rhexis and phacoemulsification and, ultimately, to fill the capsular bag.

Each solution of the set of viscoelastics has substantially different characteristics and, thus, is able to achieve different purposes. The tackiness and robustness of the first solution makes it an ideal candidate for corneal protection. However, these same characteristics make it poorly suited for the task of facilitating instrument movement. As noted above, the second of the solutions is cohesive and non-tacky, which allows for ease of instrument movement. This is important, since any hindrance on the physician's ability to operate smoothly may adversely impact the surgery, or the intended results of such surgery. Likewise, using bubble-free viscoelastic solutions will facilitate the procedure by avoiding the introduction of air bubbles into a surgical field during ophthalmic viscosurgery.

In another embodiment of the present invention, the first viscoelastic solution is used to maintain the shape of the anterior chamber while the second solution is used to maintain the shape of the capsular bag. One skilled in the art will realize that there are a variety of ways in which the present solutions may be used during viscosurgery. Additionally, a set of viscoelastics designed in accordance with the present invention could incorporate more than two viscoelastic solutions and could be designed to protect additional eye structures.

In one embodiment of the invention, the solutions are introduced one by one into the eye. Application of the solutions may be done with individual instruments. For example, a first solution may be applied with a first syringe, a second solution may be applied with a second syringe, and so forth. Alternatively, the solutions may be applied with one device. For example, a device that can separately store the multiple viscoelastic solutions may be used to preferentially administer different viscoelastic solutions to various areas of the eye during ophthalmic viscosurgery. Using such a device is more cost-effective than using separate devices to apply each of the solutions. The device may be configured with multiple compartments, one stacked upon the other, so that the solutions do not mix prior to injection. Such multi-compartment device may take the form of a multi-compartment syringe that has an angled cannula tip, such that the tip can be introduced safely into the eye. Optimally, the cornea may be pierced with a separate device.

One skilled in the art will realize that, given the differences between the viscoelastic solutions, it is possible to contain both solutions within one chamber in a distributing apparatus. That is, the solutions may contact one another without mixing. In this manner, the second and subsequent viscoelastic solution(s) may be dispensed after the bulk of the first viscoelastic solution is dispensed. Likewise, a dispensing apparatus which holds the solutions separately in a side-by-side configuration or an apparatus that dispenses the solutions through different tips is within the ambit of the invention.

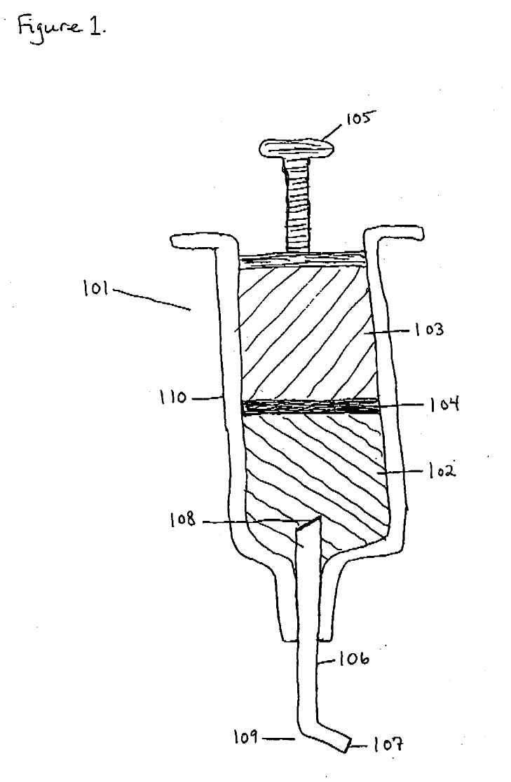

In the embodiment of the invention shown in FIG. 1, the administration device is a bi-compartment syringe 101 wherein solutions are contained in separate compartments that are situated above each other in the device, enclosed by a common housing 110. The compartments are optimally separated by a membrane or separation member 104. In this embodiment, the distal end of the bi-compartment syringe 101 may be a cannula 106 having a distal blunt end 107 that is external to the syringe body and a proximal sharp end 108 that is internal to the syringe body. Initially, the proximal sharp end 108 of the cannula 106 that is adjacent to the first viscoelastic solution 102 may be in fluid contact with the first viscoelastic solution 102 or may be separated from said first viscoelastic solution 102 by a membrane or other device. To dispense the first viscoelastic solution 102, the user applies pressure to a plunger 105 at the proximal end of the bi-compartment syringe 101. Said applied pressure forces the separation member 104 downward by fluid pressure, thus forcing the first viscoelastic solution 102 through the sharp proximal end 108 of the cannula 106 and expelling the first viscoelastic solution 102 from the distal blunt end 107 of the cannula 106. Upon application of additional pressure, the separation member 104 comes into contact with the sharp proximal end 108 of the cannula 106 and is then pierced. At this point, the cannula 106 is in fluid contact with the second viscoelastic solution 103. Continued pressure on the aforementioned plunger 105 serves to force the second viscoelastic solution 103 through the sharp proximal end 108 of the cannula 106 and expel the second viscoelastic solution 103 from the distal blunt end 107 of the cannula 106.

One skilled in the art will realize that other multi-compartment devices, including those known in the art, may be used to separately introduce multiple viscoelastic solutions into the eye for concurrent use during ophthalmic surgery. Likewise, many different separation member designs are possible in addition to the membrane described in the above embodiment. Further, one skilled in the art will realize that the present invention is not limited to only two solutions. For example, three or more viscoelastic solutions may be separately injected. Likewise, two or more viscoelastic solutions may be pre-mixed prior to administration.

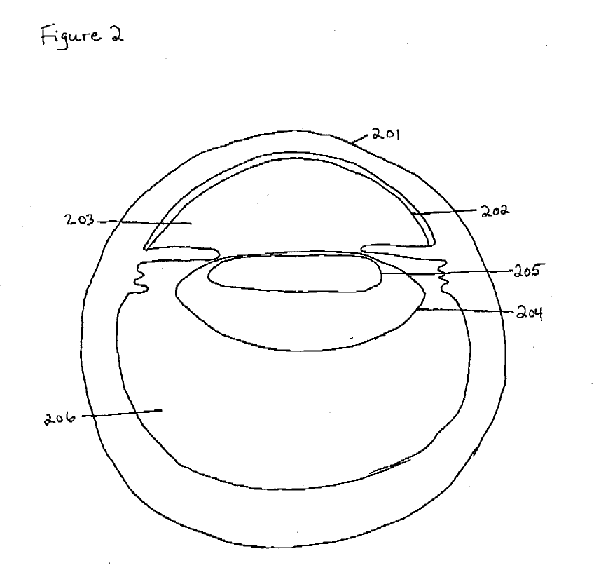

The present invention will now be discussed with reference to one particular ophthalmic viscosurgical procedure, cataract surgery, using the syringe of FIG. 1 and the cross-sectional view of the eye shown in FIG. 2 as a reference. One skilled in the art will readily realize that the present invention is similarly applicable to many other procedures. This procedure will be described using the bi-compartment syringe 101 according to the embodiment of the invention shown in FIG. 1.

One of the first steps in a cataract surgery according to the present invention is to pierce the cornea 201. Such piercing may be accomplished with any device known in the art. The first viscoelastic solution 101 is then applied to the corneal endothelium 202. To facilitate accurate placement of the first viscoelastic solution 101 against the corneal endothelium 202, the tip 109 of the bi-compartment syringe 101 may be angled. This allows the surgeon to insert the tip 109 through the pierced cornea 201 and place it adjacent to the corneal endothelium 202 without placing undue pressure on the opening in the cornea 201, and with relative ease. In alternative embodiments of the invention, syringes without angled tips may be used.

The first viscoelastic solution 102 is typically a dispersive product, having a high concentration and a low molecular weight. In one embodiment of the invention, the first viscoelastic solution 102 is tacky and robust so that it will adhere to the corneal endothelium 202. In one embodiment of the invention, the first viscoelastic solution 102 is a pure solution of sodium hyaluronate, although any other salt of hyaluronic acid may also be used. By way of example, and not of limitation, suitable salts according to the invention include magnesium, calcium or potassium salts. The first viscoelastic solution 102 may also be a pure solution of non-animal origin sodium hyaluronate. The concentration of sodium hyaluronate in the first viscoelastic solution 102 is in the range of 20-70 mg/mL, and optimally in the range of 20-40 mg/mL. The molecular weight of sodium hyaluronate in the first viscoelastic solution 102 is in the range of 50,000 daltons to 1 million daltons, and optimally less than 500,000 daltons. Vitrax™ (Allergan, Inc., Waco, Tex.) is representative of the first viscoelastic solution 102. The volume of the first viscoelastic solution 102 in the bi-compartment syringe 101 is in the range of 0.1-0.45 mL, and optimally in the range of 0.3-0.45 mL. The volume administered of the first viscoelastic solution 102 is sufficient to coat the corneal endothelium 202 without filling the anterior chamber 203 and is in the range of 0.1-0.3 mL. By way of example, the volume administered in a typical eye is in the range of 0.1-0.2 mL.

The second viscoelastic solution 103 is then applied so that it fills the remainder of the anterior chamber 203. The second viscoelastic solution 103 is a cohesive, non-tacky product, having a low concentration and a high molecular weight. The second viscoelastic solution 103 is a pure solution of sodium hyaluronate, although any other salt of hyaluronic acid may also be used. By way of example, and not of limitation, suitable salts according to the invention include magnesium, calcium or potassium salts. The second viscoelastic solution 103 may also be a pure solution of non-animal origin sodium hyaluronate. The concentration of sodium hyaluronate in the second viscoelastic solution 103 is in the range of 10-20 mg/mL and the molecular weight of sodium hyaluronate in the second viscoelastic solution 103 is greater than 1 million daltons. The volume administered of the second viscoelastic solution 103 is in the range of 0.05-0.2 mL. By way of example, the volume administered in a typical eye is in the range of 0.05-0.15 mL. Healon™ (Pharmacia, Inc., Piscataway, N.J.) is representative of the second viscoelastic solution 103.

A capsulotomy is then performed to facilitate removal of the cataract 205 from the capsular bag 204. Phacoemulsification and extraction of the cataract 205 is performed in such a manner that the first viscoelastic solution 102 stays in place and the second viscoelastic solution 103 is exchanged with a balanced salt solution (BSS), as necessary. One skilled in the art will realize that alternative cataract extraction techniques, such as endocapsular extractions, are possible. An additional volume of the second viscoelastic solution 103 is then introduced into the capsular bag 204, such that the natural shape and depth of the capsular bag 204 are restored. The additional volume administered of the second viscoelastic solution 103 is in the range of 0.05-0.4 mL. By way of example, the volume administered in a typical eye is 0.15-0.25 mL. Introduction of a new lens may then be accomplished with no damage to the cornea 201 and leaving the first viscoelastic solution 102 in place. Additional injections of the second viscoelastic solution 103 may be necessary during cataract extraction and lens implantation. After completion of the surgical procedure, the various viscoelastic compounds are removed through means known in the art.

As noted in the example of the present invention described above, the volume available in the bi-compartment syringe 101 of the first viscoelastic solution 102 is in the range of 0.1-0.45 mL, and the volume administered of the first viscoelastic solution 102 is in the range of 0.1-0.3 mL (typically in the range of 0.1-0.2 mL), such that the first viscoelastic solution 102 does not fill the entirety of the anterior chamber 203. The volume of the second viscoelastic solution 103 in the bi-compartment syringe 101 is in the range of 0.1-0.6 mL. A volume at the upper end of this range may be administered, depending upon the needs of the particular surgery. By way of example, the cumulative volume administered of the second viscoelastic solution 103 in a typical eye is in the range of 0.2-0.4 mL, such that the second viscoelastic solution 103 initially fills the remainder of the anterior chamber 203 and the capsular bag 204, and then keeps those eye structures filled with additional volumes of the second viscoelastic solution 103, as may be required during cataract extraction and lens implantation. One skilled in the art will realize that if more than two solutions are used, these volumes may change. The skilled operator will be able to calculate such changes based on the teachings disclosed herein and known dimensions of the eye.

One skilled in the art will realize that other ophthalmic surgical procedures can be practiced with the materials of the present invention. Likewise, one skilled in the art will realize that more than two viscoelastic solutions may be used during ophthalmic surgical methods that are disclosed by the present invention. For example, in cataract surgery, one viscoelastic solution can be applied to the corneal endothelium, another viscoelastic solution can be used to fill the remainder of the anterior chamber and a third viscoelastic solution can be used to fill the lens capsular bag following phacoemulsification and removal of the cataract. In ophthalmic surgical procedures employing more than two solutions, one of the solutions may be left in the eye for treatment of a particular condition. For example, the third solution may include an antibiotic, or a treating solution for the prevention of posterior capsular opacification. Further, one skilled in the art will realize that alternative embodiments of multi-compartment devices may be used to accomplish the surgical methods of the present invention.

In alternative embodiments of the invention, one or more of the viscoelastic solutions may be colored. By way of example, each solution may be given a different color. Colored solutions allow a surgeon to ensure that sufficient amounts of the solutions have been injected into the eye and also that the solutions have been removed from the eye at the end of the surgical procedure.

While the description above refers to particular embodiments of the present invention, it should be readily apparent to people of ordinary skill in the art that a number of modifications may be made without departing from the spirit thereof. The accompanying claims are intended to encompass such modifications as would fall within the true spirit and scope of the invention. The presently disclosed embodiments are, therefore, to be considered in all respects as illustrative and not restrictive. Accordingly, the scope of the invention is indicated by the appended claims rather than the foregoing description. All changes that come within the meaning and range of equivalency of the claims are intended to be embraced therein.

What is claimed is:

1. A system for performing ophthalmic viscosurgery, the system comprising:
a multi-compartment administration device comprising:
a barrel with a proximal end and a distal end;
a hollow cannula extending from the distal end of the barrel, the cannula being in flowable communication with the barrel;
a first interior compartment adjacent to the distal end of the barrel;
a second interior compartment adjacent to the proximal end of the barrel, the first and second compartments being separated by a dividing member extending across the barrel; and
a plunger coupled to the proximal end of the barrel, the plunger being configured such that, upon activation, the second compartment is brought into flowable communication with the cannula;
a first hyaluronic acid salt solution, having a concentration of hyaluronic acid salt of 20-70 mg/mL and a molecular weight of hyaluronic acid salt of 50,000-1,000,000 daltons; and
a second hyaluronic acid salt solution having a concentration of hyaluronic acid salt of 10-20 mg/mL and a molecular weight of hyaluronic acid salt of greater than 1,000,000 daltons.

2. The system for performing ophthalmic viscosurgery of claim 1, wherein the dividing member is formed from a compound selected from the group consisting of silicone rubber and buthyl rubber.

3. The system for performing ophthalmic viscosurgery of claim 1, wherein the volume administered of the first hyaluronic acid salt solution is in the range of 0.1-0.3 mL.

4. The system for performing ophthalmic viscosurgery of claim 1, wherein the volume administered of the second hyaluronic acid salt solution is in the range of 0.2-0.4 mL.

5. The system for performing ophthalmic viscosurgery of claim 1, wherein the cannula has an angled tip.

6. The system for performing ophthalmic viscosurgery of claim 1, wherein the cannula has a sharp, proximal end and a blunt, distal end.

7. The system for performing ophthalmic viscosurgery of claim 1, wherein one of the first and second hyaluronic acid salt solutions is colored.

8. The system for performing ophthalmic surgery of claim 1, wherein the hyaluronic acid salt is selected from the group consisting of sodium, magnesium, calcium and potassium hyaluronic acid salts.

9. A system for performing ophthalmic viscosurgery, the system comprising:
a multi-compartment syringe comprising:
an elongated, tubular syringe barrel with a proximal end and a distal end;
a hollow cannula extending from the distal end of the syringe barrel, the cannula being in flowable communication with the syringe barrel, the cannula having a sharp, proximal end and a blunt, distal end;
a first interior compartment adjacent to the distal end of the syringe barrel, and in flowable communication with the cannula;
a second interior compartment adjacent to the proximal end of the syringe barrel, the first and second compartments being separated by a dividing member extending across the barrel; and
a plunger coupled to the proximal end of the syringe barrel, the plunger being configured such that, upon activation, the dividing member is pierced, bringing the second compartment into flowable communication with the cannula;
a first bubble-free, tacky hyaluronic acid salt solution, having a concentration of hyaluronic acid salt of 20-70 mg/mL and a molecular weight of hyaluronic acid salt of 50,000-1,000,000 daltons; and
a second bubble-free, non-tacky hyaluronic acid salt solution, having a concentration of hyaluronic acid salt of 10-20 mg/mL and a molecular weight of hyaluronic acid salt of greater than 1,000,000 daltons.

10. The system for performing ophthalmic viscosurgery of claim 9, wherein the dividing member is formed from a compound selected from the group consisting of silicone rubber and buthyl rubber.

11. The system for performing ophthalmic viscosurgery of claim 9, wherein the volume administered of the first hyaluronic acid salt solution is in the range of 0.1-0.3 mL.

12. The system for performing ophthalmic viscosurgery of claim 9, wherein the volume administered of the second hyaluronic acid salt solution is in the range of 0.2-0.4 mL.

13. The system for performing ophthalmic viscosurgery of claim 9, wherein the cannula has an angled tip.

14. The system for performing ophthalmic viscosurgery of claim 9, wherein one of the first and second viscoelastic solutions is colored.

15. The system for performing ophthalmic surgery of claim 9, wherein the hyaluronic acid salt is selected from the group consisting of sodium, magnesium, calcium and potassium hyaluronic acid salts.

16. A method of performing ophthalmic viscosurgery on an eye having a cornea, a corneal endothelium, an anterior chamber, a lens capsular bag, a lens and a posterior chamber, the method comprising the steps of:
providing a dispensing device containing first and second viscoelastic solutions, wherein the volume of the first solution is less than the volume of the anterior chamber of the eye;
piercing the cornea;
applying the first viscoelastic solution to the corneal endothelium;
introducing a first portion of the second viscoelastic solution into the anterior chamber of the eye;
performing a cataract extraction;
introducing a second portion of the second viscoelastic solution into the lens capsular bag; and
implanting a new lens.

17. The method of performing ophthalmic viscosurgery of claim 16, wherein the dispensing device is configured to dispense the first viscoelastic solution through a cannula having an angled tip.

18. The method of performing ophthalmic viscosurgery of claim 16, wherein the introduction of the first portion of the second viscoelastic solution fills the anterior chamber of the eye.

19. The method of performing ophthalmic viscosurgery of claim 16, wherein the volume administered of the first viscoelastic solution is in the range of 0.1-0.3 mL.

20. The method of performing ophthalmic viscosurgery of claim 16, wherein the introduction of the second portion of the second viscoelastic solution fills the lens capsular bag of the eye.

21. The method of performing ophthalmic viscosurgery of claim 16, further including the step of performing a capsulotomy.

22. The method of performing ophthalmic viscosurgery of claim 16, further including the step of removing substantially all of the first and second viscoelastic solutions from the eye.

23. A method of performing ophthalmic viscosurgery on an eye having a cornea, a corneal endothelium, an anterior chamber, a lens capsular bag, a lens and a posterior chamber, the method comprising the steps of:
providing a multi-compartment syringe containing first and second viscoelastic solutions in separate compartments, wherein the volume of the first solution is less than the volume of the anterior chamber of the eye, the syringe having a tubular barrel with a proximal end and a distal end, a hollow cannula in flowable communication with the syringe extending from the distal end of the syringe barrel, a first interior compartment adjacent to the distal end of the barrel, a second interior compartment adjacent to the proximal end of the barrel, the first and second compartments being separated by a dividing member extending across the barrel, and a plunger coupled to the proximal end of the syringe barrel, the plunger being configured such that, upon activation, the second compartment is brought into flowable communication with the cannula;
piercing the cornea;
applying the first viscoelastic solution to the corneal endothelium;
introducing a first portion of the second viscoelastic solution into the anterior chamber of the eye;
performing a cataract extraction;
introducing a second portion of the second viscoelastic solution into the lens capsular bag; and
implanting a new lens.

24. The method of performing ophthalmic viscosurgery of claim 23, wherein the multi-compartment syringe is configured to dispense the first viscoelastic solution through a cannula having an angled tip.

25. The method of performing ophthalmic viscosurgery of claim 23, wherein the introduction of the first portion of the second viscoelastic solution fills the anterior chamber of the eye.

26. The method of performing ophthalmic viscosurgery of claim 23, wherein the volume administered of the first viscoelastic solution is in the range of 0.1-0.3 mL.

27. The method of performing ophthalmic viscosurgery of claim 23, wherein the introduction of the second portion of the second viscoelastic solution fills the lens capsular bag of the eye.

28. The method of performing ophthalmic viscosurgery of claim 23, further including the step of performing a capsulotomy.

29. The method of performing ophthalmic viscosurgery of claim 23, further including the step of removing substantially all of the first and second viscoelastic solutions from the eye.
